(19) United States
(12) Patent Application Publication
(10) Pub. No.: US 2005/0101593 A1
(43) Pub. Date: May 12, 2005

(54) Phosphoric acid salt of an integrin receptor antagonist

(76) Inventors: Robert S Meissner, Schwenksville, PA (US); Wei Xu, North Wales, PA (US)

Correspondence Address: MERCK AND CO., INC, P O BOX 2000, RAHWAY, NJ, 07065-0907, US

(21) Appl. No.: 10/398,366

(22) PCT Filed: Oct. 1, 2001
(86) PCT No.: PCT/US01/30647

(30) Foreign Application Priority Data
Oct. 4, 2000 (US) 60237534

(51) Int. Cl. A61K 31/55; C07D 487/02

(52) U.S. Cl. 514/215; 540/577

(57) ABSTRACT

The phosphoric acid salt of 3-(2-methoxy-pyrimidin-5-yl)-5-oxo-9-(6,7,8,9-tetrahydro-5H-pyrido[2,3-b]azepin-2-yl)-nonanoic acid is a potent antagonist of the integrin αvβ3 receptor and is useful for the prevention and/or treatment of osteoporosis and vascular restenosis, as well as conditions associated with excessive angiogenesis, such as macular degeneration, diabetic retinopathy, atherosclerosis, inflammatory arthritis, cancer, and metastatic tumor growth. The invention also relates to a process for the preparation of the novel salt as well as pharmaceutical compositions and methods of use.

### FIELD OF THE INVENTION

The present invention relates to a particular salt of an integrin receptor antagonist. More particularly, the invention relates to a phosphoric acid salt of 3-(2-methoxy-pyrimidin-5-yl)-5-oxo-9-(6,7,8,9-tetrahydro-5H-pyrido[2,3-b]azepin-2-yl)-nonanoic acid, which is a potent integrin αᵥβ₃ receptor antagonist. This novel salt is therefore useful for the treatment and prevention of diseases and conditions for which an antagonist of the integrin αᵥβ₃ receptor is indicated.

### BACKGROUND OF THE INVENTION

Integrin αᵥβ₃ receptor antagonists have been described as being of use for the prevention and/or treatment of osteoporosis, vascular restenosis, macular degeneration, diabetic retinopathy, atherosclerosis, inflammatory arthritis, cancer, and metastatic tumor growth [see, for example, M. E. Duggan, et al., “Ligands to the integrin receptor αᵥβ₃, Exp. Opin. Ther. Patents, 10: 1367-1383 (2000); M. Gowen, et al., “Emerging therapies for osteoporosis,” Emerging Drugs, 5: 1-43 (2000); J. S. Kerr, et al., “Small molecule αᵥ integrin antagonists: novel anticancer agents,” Exp. Opin. Invest. Drugs, 9: 1271-1291 (2000); and W. H. Miller, et al., “Identification and in vivo efficacy of small-molecule antagonists of integrin αᵥβ₃ (the vitronectin receptor),” Drug Discovery Today, 5: 397-408 (2000)].

U.S. Ser. No. 09/583,522, assigned to Merck & Co., describes a class of chain-oxidized 9-substituted-3-aryl-nonanoic acid derivatives, which are potent integrin αᵥβ₃ receptor antagonists and therefore useful for inhibiting bone resorption, vascular restenosis, treating and/or preventing osteoporosis, and inhibiting diseases and conditions associated with excessive and undesirable angiogenesis. Specifically disclosed in U.S. Ser. No. 09/583,522 is 3-(2-methoxy-pyrimidin-5-yl)-5-oxo-9-(6,7,8,9-tetrahydro-5H-pyrido[2,3-b]azepin-2-yl)-nonanoic acid. Pharmaceutically acceptable salts of this compound are generically encompassed within the scope of U.S. Ser. No. 09/583,522.

However, there is no specific disclosure in the above reference of the newly discovered phosphoric acid salt of 3-(2-methoxy-pyrimidin-5-yl)-5-oxo-9-(6,7,8,9-tetrahydro-5H-pyrido[2,3-b]azepin-2-yl)-nonanoic acid of structural formula I below.

### SUMMARY OF THE INVENTION

This invention provides a new phosphoric acid salt of 3-(2-methoxy-pyrimidin-5-yl)-5-oxo-9-(6,7,8,9-tetrahydro-5H-pyrido[2,3-b]azepin-2-yl)-nonanoic acid of the following structural formula I: 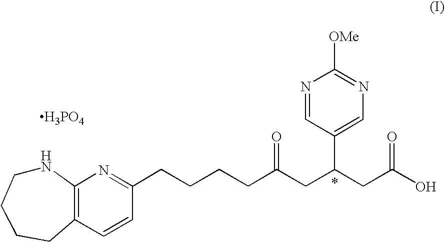
or a pharmaceutically acceptable solvate, including hydrate, thereof.

The phosphoric acid salt of the present invention has a chiral center (indicated with an *) at the C-3 position of the nonanoic acid chain and can thus occur as a racemate, racemic mixture, and single enantiomers, with all isomeric forms being included in the present invention. The separate enantiomers, substantially free of the other, are included within the scope of the invention, as well as mixtures of the two enantiomers.

Therefore, one embodiment of the present invention provides the phosphoric acid salt of 3(S)-(2-methoxy-pyrimidin-5-yl)-5-oxo-9-(6,7,8,9-tetrahydro-5H-pyrido[2,3-b]azepin-2-yl)-nonanoic acid of structural formula II: 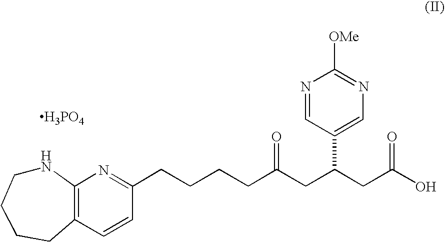
and a second embodiment of the present invention provides the phosphoric acid salt of 3(R)-(2-methoxy-pyrimidin-5-yl)-5-oxo-9-(6,7,8,9-tetrahydro-5H-pyrido[2,3-b]azepin-2-yl)-nonanoic acid of structural formula III:

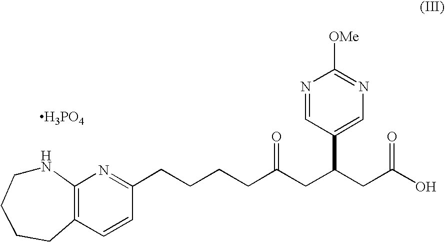

More specifically, the phosphoric acid salt of the present invention is comprised of one molar equivalent of mono-protonated 3-(2-methoxy-pyrimidin-5-yl)-5-oxo-9-(6,7,8,9-tetrahydro-5H-pyrido[2,3-b]azepin-2-yl)-nonanoic acid cation and one molar equivalent of dihydrogenphosphate (biphosphate) anion.

In a further embodiment of the present invention, the phosphoric acid salt of structural formulae I-III is crystalline.

The crystalline phosphoric acid salt of the present invention exhibits greater chemical stability to heat and light than the parent zwitterionic compound of structural formula (IV) below, which exists as an amorphous form. Moreover, the crystalline salt is easier to handle and process because it is significantly less hygroscopic than the amorphous zwitterion. The enhanced stability and non-hygroscopic nature of the crystalline salt constitute advantageous properties in the preparation of solid pharmaceutical dosage forms containing the pharmacologically active ingredient.

The phosphoric acid salt of the present invention, which exhibits potent integrin αᵥβ₃ antagonist activity, is particularly useful for inhibiting bone resorption, treating and/or preventing osteoporosis, and inhibiting vascular restenosis, diabetic retinopathy, macular degeneration, atherosclerosis, inflammatory arthritis, cancer, and metastatic tumor growth.

### BRIEF DESCRIPTION OF THE FIGURES

FIG. 1 is a characteristic X-ray diffraction pattern of the crystalline phosphoric acid salt of Formula I.

FIG. 2 is a differential scanning calorimetric (DSC) curve of the crystalline phosphoric acid salt of Formula I.

FIG. 3 is a carbon-13 cross-polarization magic-angle spinning (CPMAS) nuclear magnetic resonance (NMR) spectrum of the crystalline phosphoric acid salt of Formula I.

### DETAILED DESCRIPTION OF THE INVENTION

The present invention provides a pharmaceutical composition comprising the phosphoric acid salt of Formula I above, or a pharmaceutically acceptable solvate thereof, in association with one or more pharmaceutically acceptable carriers.

The compositions in accordance with the invention are suitably in unit dosage forms such as tablets, pills, capsules, powders, granules, sterile solutions or suspensions, metered aerosol or liquid sprays, drops, ampoules, auto-injector devices or suppositories. The compositions are intended for oral, parenteral, intranasal, sublingual, or rectal administration, or for administration by inhalation or insufflation. Formulation of the compositions according to the invention can conveniently be effected by methods known from the art, for example, as described in Remington's Pharmaceutical Sciences, 17^{th} ed., 1995.

The dosage regimen is selected in accordance with a variety of factors including type, species, age, weight, sex and medical condition of the patient; the severity of the condition to be treated; the route of administration; and the renal and hepatic function of the patient. An ordinarily skilled physician, veterinarian, or clinician can readily determine and prescribe the effective amount of the drug required to prevent, counter or arrest the progress of the condition.

Oral dosages of the present invention, when used for the indicated effects, will range between about 0.01 mg per kg of body weight per day (mg/kg/day) to about 100 mg/kg/day, preferably 0.01 to 10 mg/kg/day, and most preferably 0.1 to 5.0 mg/kg/day. For oral administration, the compositions are preferably provided in the form of tablets containing 0.01, 0.05, 0.1, 0.5, 1.0, 2.5, 5.0, 10.0, 15.0, 25.0, 50.0, 100 and 500 milligrams of the active ingredient for the symptomatic adjustment of the dosage to the patient to be treated. A medicament typically contains from about 0.01 mg to about 500 mg of the active ingredient, preferably, from about 1 mg to about 100 mg of active ingredient. Intravenously, the most preferred doses will range from about 0.1 to about 10 mg/kg/minute during a constant rate infusion. Advantageously, the salt of the present invention may be administered in a single daily dose, or the total daily dosage may be administered in divided doses of two, three or four times daily. Furthermore, the salt of the present invention can be administered in intranasal form via topical use of suitable intranasal vehicles, or via transdermal routes, using those forms of transdermal skin patches well known to those of ordinary skill in the art. To be administered in the form of a transdermal delivery system, the dosage administration will, of course, be continuous rather than intermittent throughout the dosage regimen.

In the methods of the present invention, the salt herein described in detail can form the active ingredient, and is typically administered in admixture with suitable pharmaceutical diluents, excipients or carriers (collectively referred to herein as ‘carrier’ materials) suitably selected with respect to the intended form of administration, that is, oral tablets, capsules, elixirs, syrups and the like, and consistent with conventional pharmaceutical practices.

For instance, for oral administration in the form of a tablet or capsule, the active drug component can be combined with an oral, non-toxic, pharmaceutically acceptable, inert carrier such as lactose, starch, sucrose, glucose, methyl cellulose, magnesium stearate, dicalcium phosphate, calcium sulfate, mannitol, sorbitol and the like; for oral administration in liquid form, the oral drug component can be combined with any oral, non-toxic, pharmaceutically acceptable inert carrier such as ethanol, glycerol, water and the like. Moreover, when desired or necessary, suitable binders, lubricants, disintegrating agents and coloring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include sodium oleate, sodium stearate, magnesium stearate, sodium benzoate, sodium acetate, sodium chloride and the like. Disintegrators include, without limitation, starch, methyl cellulose, agar, bentonite, xanthan gum and the like.

The salt of Formula I has been found to possess a high solubility in water, rendering it especially amenable to the preparation of formulations, in particular intranasal formulations, which require relatively concentrated aqueous solutions of active ingredient. The solubility of the salt of formula I in water and buffered solutions between pH 2 and 8 has been found to be greater than 12 mg/mL.

According to a further aspect, the present invention provides a process for the preparation of the phosphoric acid salt of formula I, which process comprises reacting 3-(2-methoxy-pyrimidin-5-yl)-5-oxo-9-(6,7,8,9-tetrahydro-5H-pyrido[2,3-b]azepin-2-yl)-nonanoic acid of structural formula IV below: 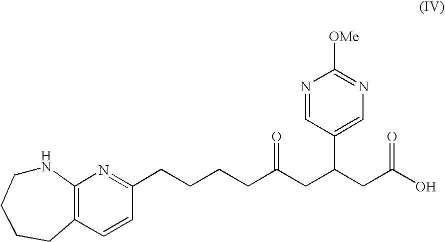
with approximately one molar equivalent of phosphoric acid in a suitable organic solvent. The process is carried out generally at about 25-100° C., and usually at about 50-80° C.

The process can be conducted by slowly adding phosphoric acid, in a concentration of about 0.1 to 19 M, to about one molar equivalent of compound of formula IV dissolved in an organic solvent at about 0-25° C. Generally, the organic solvent is a C₁-C₄ alkanol, for example, methanol, ethanol, or isopropanol. Alternatively, the process can be conducted by adding compound of formula IV dissolved in an organic solvent to about one molar equivalent of the phosphoric acid in an aqueous organic solvent at a temperature of about 25° C.

The process is advantageously carried out after the initial mixing of the reactants by warming to a temperature of about 50-80° C., then allowing the mixture to cool to room temperature, followed by aging the mixture at room temperature for about 12-24 hours. The phosphoric salt is then isolated and purified by conventional procedures.

The phosphoric acid salt of formula I can also be prepared by salt exchange, which comprises treating a salt of the compound of formula IV above, other than the 1:1 phosphoric acid salt of formula I, with a suitable dihydrogenphosphate salt. Examples of appropriate dihydrogenphosphate salts which may be utilized in the above salt exchange include metal dihydrogenphosphates, such as sodium or potassium dihydrogenphosphate, and ion-exchange resins. The reaction is conveniently carried out in an aqueous medium.

The starting compound of structural formula IV can be prepared by the procedure detailed in Schemes 1-3 and Example 1 below.

In a still further aspect, the present invention provides a method for the treatment and/or prevention of clinical conditions for which an integrin αᵥβ₃ receptor antagonist is indicated, which method comprises administering to a patient in need of such prevention or treatment a prophylactically or therapeutically effective amount of the salt of Formula I as defined above or a pharmaceutically acceptable solvate thereof.

The present invention also provides the use of the salt of Formula I as defined above or pharmaceutically acceptable solvate thereof for the manufacture of a medicament for the prevention and/or treatment of clinical conditions for which an antagonist of the integrin αᵥβ₃ receptor is indicated.

The following non-limiting Examples are intended to illustrate the present invention and should not be construed as being limitations on the scope or spirit of the instant invention.

Compounds described herein may exist as tautomers such as keto-enol tautomers. The individual tautomers as well as mixtures thereof are encompassed with compounds of structural formula I.

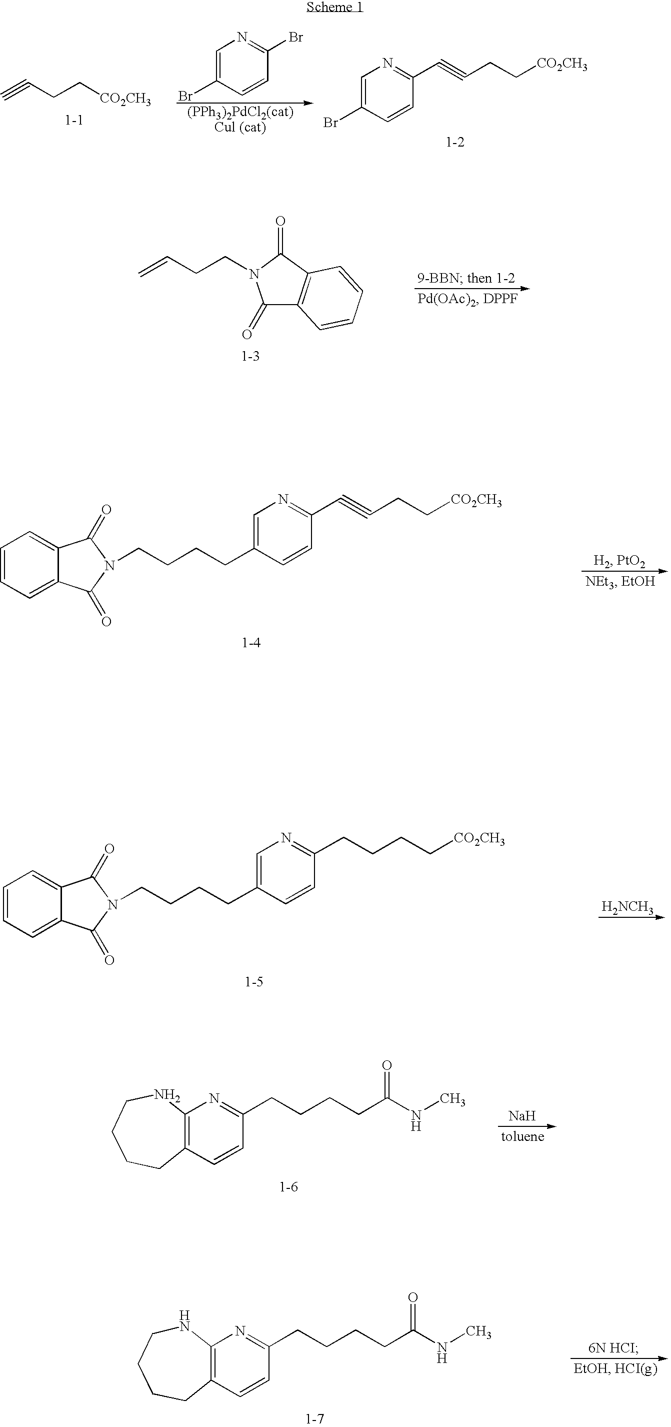

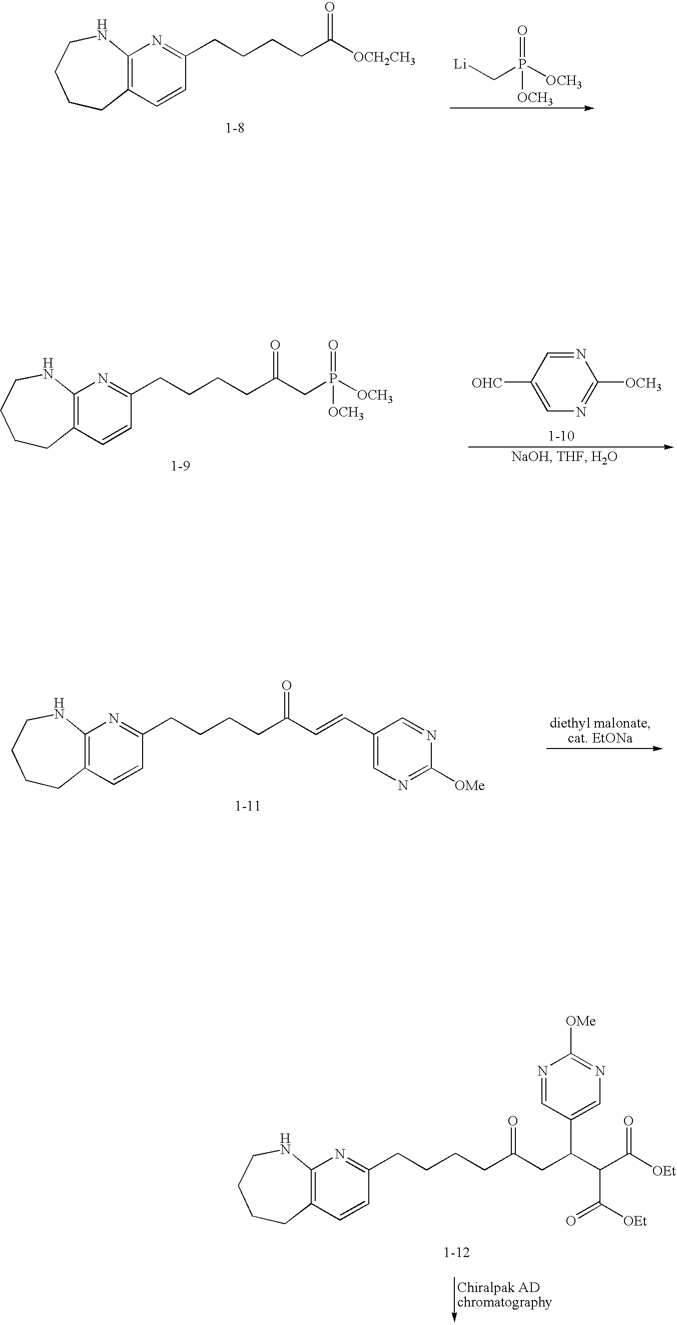

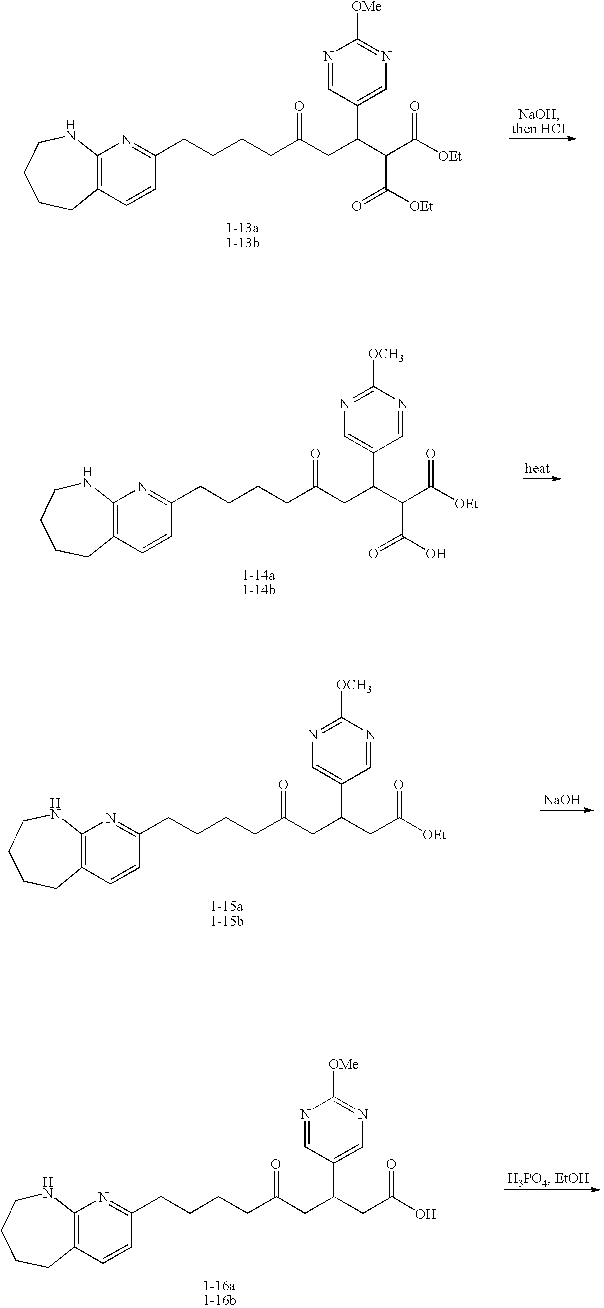

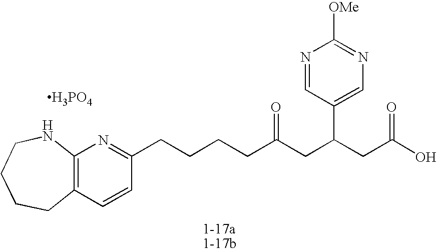

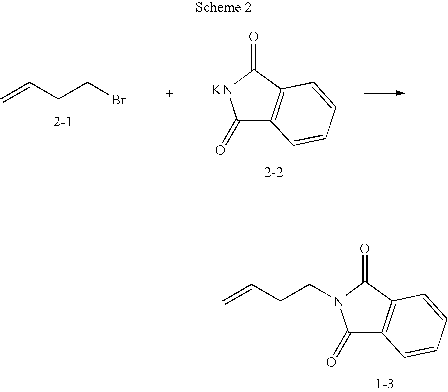

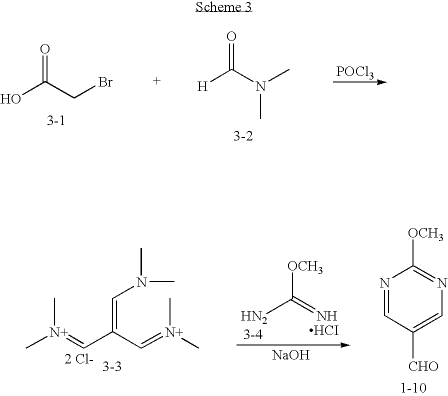

### EXAMPLE 1

### 3(S or R)-(2-Methoxy-pyrimidin-5-yl)-5-oxo-9-(6,7,8,9-tetrahydro-5H-pyrido[2,3-b]azepin-2-yl)-nonanoic Acid Phosphoric Acid Salt (1-17a)

### 5-(5-Bromo-pyridin-2-yl)-pent-4-ynoic Acid Methyl Ester (1-2)

Through a mixture of 1-1 (45.9 g, 0.409 mol), 2,5-dibromopyridine (109.07 g, 0.372 mmol), copper(I) iodide (1.77 g, 9.3 mmol), and triethylamine (1.0 L) was bubbled a stream of argon for 30 minutes. Bis(triphenylphosphino)palladium dichloride (6.52 g, 9.3 mmol) was then added, and the mixture heated at 60° C. for 6 hours, then aged at ambient temperature for 12 hours. The mixture was diluted with ether, washed with water and brine, dried over magnesium sulfate, and concentrated to an oil. The residue was loaded onto a silica gel column as a solution in dichloromethane, and eluted with 10% to 40% EtOAc/hexanes to give 1-2 (79.3 g, 79%).

TLC R_{f}=0.41 (silica, 40% EtOAc/hexanes).

¹H NMR (400 MHz, CDCl₃) δ 8.60 (s, 1H), 7.76 (d, 1H), 7.24 (d, 1H), 3.73 (s, 3H), 2.76 (m, 2H), 2.63 (m, 2H).

### 2-But-3-enyl-isoindole-1,3-dione (1-3)

To a stirred solution of 4-bromo-1-butene (2-1, 20 g, 148 mmol) in DMF (150 mL) was added potassium phthalimide (2-2, 25 g, 133 mmol) and the mixture stirred for 18 hours at 70° C. After cooling to room temperature, the mixture was diluted with ether, washed with water and brine, dried over MgSO₄, and concentrated to give 1-3 as a white solid (29.8 g, 86%).

¹H NMR (400 MHz, CDCl₃) a 7.85 (m, 2H), 7.72 (m, 2M), 5.82 (m, 1H), 5.08 (m, 2H), 3.77 (t, 2H, J=7 Hz), 2.44 (m, 2H).

### 5-{5-[4-(1,3-Dioxo-1,3-dihydro-isoindol-2-yl)-butyl]-pyridin-2-yl}-pent-4-ynoic Acid Methyl Ester (1-4)

To solid 1-3 (77 g, 0.383 mol) was added a solution of 9-BBN in THF (795 mL, 0.5 M, 0.398 mmol), and the resulting yellow solution stirred for 15 hours at ambient temperature. To this solution was then added potassium carbonate powder (81 g, 0.59 mol) and 1-2 (79 g, 0.295 mol), and the resulting mixture degassed with argon bubbling for 30 minutes. To this mixture was then added a premixed, degassed, aged (70° C. for 40 minutes with vigorous stirring) suspension of Pd(OAc)₂ (9-9 g, 44 mmol) and DPPF (25 g, 44 mmol) in DMF (350 mL) via a cannula. The mixture was then heated at reflux (70° C.) for 6 hours. Following cooling, the mixture was reduced to a thick slurry, diluted with ether, washed with water and brine. The combined aqueous layers were extracted once with ethyl acetate, and the combined organics were dried over MgSO₄ and concentrated to give a brown oil. The oil was diluted with xylenes and evaporated to give a brown solid. This residue was chromatographed on silica gel (25-75% EtOAc/hexanes) to give 1-4 (89.9 g, 81%) as a tan solid.

TLC R_{f}=0.28 (silica, 50% EtOAc/hexanes).

¹H NMR (400 MHz, CDCl₃) δ 8.36 (s, 1H), 7.84 (m, 2H), 7.67 (m, 2H) 7.42 (m, 1H), 7.23 (m, 1H), 3.71 (m, 5M), 2.78 (m, 2H), 2.63 (m, 4H), 1.66 (m, 4H).

### 5-{5-[4-(1,3-Dioxo-1,3-dihydro-isoindol-2-yl)-butyl]-pyridin-2-yl}-pentanoic Acid Methyl Ester (1-5)

A mixture of 1-4 (90 g, 0.239 mol), triethylamine (33 mL, 0.239 mol), and PtO₂ (8 g) was stirred under a balloon of hydrogen for 6 hours. The mixture was filtered through celite, the cake washed with methanol, and the filtrate evaporated to gave 1-5 (91 g, 97%).

TLC R_{f}=0.28 (silica, 50% EtOAc/hexanes).

¹H NMR (400 MHz, CDCl₃) δ 8.36 (s, 1H), 7.82 (m, 2H), 7.65 (m, 2H) 7.40 (m, 1H), 7.04 (m, 1H), 3.75 (m, 3H), 3.64 (s, 3H), 2.78 (t, 2H), 2.62 (t, 2H), 2.38 (t, 2H), 1.71 (m, 8H).

### 5-[5-(4-Amino-butyl)-pyridin-2-yl]-pentanoic Acid Methylamide (1-6)

A solution of 1-5 (31 g, 79 mmol) in a saturated solution of methylamine in methanol (500 mL) was divided among three 350 mL glass pressure bottles, and each was heated at 80° C. for 15 hours. The mixture was cooled, combined, and concentrated to an oil under reduced pressure with the bath temperature not exceeding 25° C. The residue was chromatographed on silica gel (10:10:1:1 EtOAc/EtOH/NH₄OH/H₂O) to give 1-6 as a yellow oil (18.5 g, 86%).

TLC R_{f}=0.16 (silica, 10:10:1:1 EtOAc/EtOH/NH₄OH/H₂O).

¹H NMR (400 MHz, CDCl₃) δ 8.32 (s, 1H), 7.41 (m, 1H), 7.07 (m, 1H), 2.74 (m, 7H), 2.59 (t, 2H, J=6 Hz), 2.21 (t, 2H, J=6 Hz), 1.69 (m, 6H), 1.48 (m, 2H).

### 5-(6,7,8,9-Tetrahydro-5H-pyrido[2,3-b]azepin-2-yl)-pentanoic Acid Methylamide (1-7)

A mixture of 16 (24 g, 91.2 mmol) and NaH (10.9 g of a 60% weight dispersion in mineral oil, 273 mmol) in xylenes (500 mL) was purged with argon for 30 min, and then heated at reflux for 72 hours. The mixture was cooled, quenched with ethanol, diluted with 10% aqueous potassium carbonate and extracted with ethyl acetate. The organics were dried over MgSO₄ and concentrated to an oil. The residue was chromatographed on silica gel (70:25:5 CHCl₃/EtOAc/MeOH/H₂O) to give 1-7 as a white solid (10.5 g, 44%).

TLC R_{f}=0.15 (silica, 70:25:5 CHCl₃/EtOAc/MeOH).

¹H NMR (400 MHz, CDCl₃) δ 7.24 (d, 1H, J=7 Hz), 6.53 (d, 1H, J=7 Hz), 5.43 (br s, 1H), 4.62 (br s, 1H), 3.12 (m, 2H), 2.79 (d, 3H, J=5 Hz), 2.63 (m, 4H), 2.18 (m, 2H), 1.81 (m, 2H), 1.68 (m, 6 Hz).

### 5-(6,7,8,9-Tetrahydro-5H-pyrido[2,3-b]azepin-2-yl)-pentanoic Acid Ethyl Ester (1-8)

A mixture of 1-7 (3 g, 11.5 mmol) and 6 M HCl (100 mL) in a sealed tube was heated at 70° C. for 12 hours. The mixture was cooled and concentrated to an oil. The residue was azeotroped from ethanol (50 mL) twice, then dissolved in 4 M HCl in ethanol (100 mL) and heated at 70° C. for 1 hour. The mixture was cooled and concentrated to an oil. The residue was diluted with ethyl acetate, washed with 10% aqueous potassium carbonate and brine, dried over MgSO₄, and concentrated to give 1-8 (2.9 g, 92%) as an oil.

TLC R_{f}=0.44 (silica, 70:25:5 CHCl₃/EtOAc/MeOH).

¹H NMR (400 MHz, CDCl₃) δ 7.22 (d, 1H, J=7 Hz), 6.53 (d, 1H, J=7 Hz), 4.63 (br s, 1H), 4.11 (q, 2H, J=7 Hz), 3.12 (m, 2H), 2.66 (m, 2H), 2.62 (t, 2H, J=6 Hz), 2.33 (t, 2H, J=6 Hz), 1.70 (m, 2H), 1.63 (m, 6H), 1.27 (t, 3H, J=7 Hz).

### [2-Oxo-6-(6,7,8,9-tetrahydro-5H-pyrido[2,3-b]azepin-2-yl)-hexyl]-phosphonic Acid Dimethyl Ester (1-9)

To a solution of dimethyl methylphosphonate (38 g, 0.30 mmol) in THF at −78° C. was added n-butyllithium (112 mL of a 2.5 M solution in hexanes, 0.28 mol) over 20 minutes. After an additional 40 minutes, the ester 1-8 (22.8 g, 82.5 mmol) was added in 100 mL THF over 15 minutes. After stirring for 30 minutes, methanol (25 mL) was added, followed by saturated aqueous ammonium chloride (200 mL), and the mixture allowed to warm to ambient temperature. The mixture was reduced to one-fifth its original volume by evaporation, then diluted with ethyl acetate, and washed with water and brine. The combined aqueous layers were extracted with ethyl acetate, and the combined organics dried over MgSO₄ and evaporated to give 1-9 (27.8 g, 95%) as an oil.

TLC R_{f}=0.19 (silica, 70:25:5 CHCl₃/EtOAc/MeOH).

¹H NMR (400 MHz, CDCl₃) δ 7.22 (d, 1H, J=7 Hz), 6.57 (d, 1H, J=7 Hz), 4.63 (br s, 1H), 3.78 (d, 6H, J=11 Hz), 3.17 (m, 2H), 3.10 (d, 2H, J=23 Hz), 2.63 (m, 6H), 1.80 (m, 2H), 1.67 (m, 6H).

### 2-Methoxy-pyrimidine-5-carboxaldehyde (1-10)

To a solution of bromoacetic acid 3-1 (12 g, 86.4 mmol) in DMF (44 mL) at 90° C. was added phosphorous oxychloride (24 mL, 260 mmol) over 5 h and then heated to 110° C. After stirring at 110° C. for 2.5 h, the mixture was cooled to 45° C. and quenched into a cold isopropanol (44 mL) at 2° C. and diluted with isopropyl acetate (44 mL) and then treated with water (6.2 mL), which was added over 45 minutes at 2° C. to form the dichloride vinamidinium salt 3-3. After stirring for 1 h, the deposited solid was collected and washed with isopropyl acetate (2×14 mL) and acetonitrile (2×14 mL) to afford 3-3 (12.0 g, 54%) as a pale yellow crystal.

To a slurry mixture of dichloride vinamidinium salt 3-3 (10.1 g, 39.9 mmole) and acetamidine hydrochloride 3-4 (4.2 g, 44.4 mmol) in acetonitrile (48 mL) at 22° C. was added 50% sodium hydroxide (4.9 g, 61.1 mmol) over 1.5 h and stirred at room temperature for 1.5 h.

The reaction mixture was filtered and washed with acetonitrile (10 mL), and the combined filtrate was concentrated under reduced pressure and solvent switched to heptane. The resulting heptane slurry mixture of crude 1-10 (25 mL) was extracted with methyl t-butyl ether (MTBE) (4×20 mL) at 40° C. The combined MTBE extract was filtered through a pad of fine silica gel and concentrated under reduced pressure. The residue was recrystallized from heptane to give aldehyde 1-10 (2.15 g, 44%) as pale yellow solid; m.p. 78-79° C.

¹H NMR (400 MHz, CDCl₃): δ 10.1 (s, 1H), 9.04 (s, 2H), 2.80 (s, 3H) ppm.

### 1-(2-Methoxy-pyrimidin-5-yl)-7-(6,7,8,9-tetrahydro-5H-pyrido[2,3-b]azepin-2-yl)-hept-1-en-3-one (1-11)

To a solution of 1-10 (8.99 g, 64 mmol) and 1-9 (22 g, 63 mmol) in THF (300 mL) at 0° C. was added NaOH (17 mL of a 4N solution in water, 69.2 mmol) over 20 minutes. After an additional 15 minutes, the mixture was allowed to warm to ambient temperature and stir for 30 minutes, then concentrated to ⅕ volume. The residue was diluted with ethyl acetate, washed with 5% w/v aqueous potassium carbonate and brine, dried over magnesium sulfate, and concentrated to give 1-11 (22.5 g, 98%) as a yellow solid.

¹H NMR (400 MHz, CDCl₃) δ 8.68 (s, 2H), 7.42 (d, 1H, J=11 Hz), 7.22 (d, 1H, J=7 Hz), 6.76 (d, 1H, J=1 Hz), 6.58 (d, 1H, J=7 Hz), 4.62 (br s, 1H), 4.03 (s, 3H), 3.11 (m, 2H), 2.62 (m, 6H), 1.80 (m, 2H), 1.73 (m, 6H).

### 2-[1(S or R)-(2-Methoxy-pyrimidin-5-yl)-3-oxo-7-(6,7,8,9-tetrahydro-5H-pyrido[2,3-b]azepin-2-yl)-heptyl]-malonic Acid Diethyl Ester (1-13a)

To a solution of 1-11 (22.5 g, 62 mmol) and diethyl malonate (11.9 mL, 74.4 mmol) in ethanol (150 mL) and THF (150 mL) was added sodium ethoxide (0.2 mL of a 21% w/w solution in ethanol). After 3 hr, HPLC showed complete consumption of the starting materials. The mixture was concentrated, and the residue purified in one injection on a 10×50 cm Chiralpak AD column (flow=275 mL/min, A:B=30:70) (A=0.1% diethylamine/hexane, B=2-propanol). Product 1-13a eluted at about 30 minutes, providing 14.0 g of 1-13a (96% yield); its enantiomer, 1-13b, eluted at about 60 minutes.

¹H NMR (400 MHz, CDCl₃) δ 8.42 (s, 2H), 7.21 (d, 1H, J=7 Hz), 6.49 (d, 1H, J=7 Hz), 4.61 (br s, 1H), 4.20 (m, 2H), 4.03 (m, 2H), 3.98 (s, 3H), 3.92 (m, 1H), 3.70 (m, 2H), 3.11 (m, 2H), 2.92 (m, 2H), 2.62 (m, 2H), 2.53 (m, 2H), 2.39 (m, 2H), 1.79 (m, 2H), 1.68 (m, 2H), 1.58 (m, 4H), 1.26 (m, 3H), 1.11 (t, 3H).

### 2-[1(S or R)-(2-Methoxy-pyrimidin-5-yl)-3-oxo-7-(6,7,8,9-tetrahydro-5H-pyrido[2,3-b]azepin-2-yl)-heptyl]-malonic Acid Monoethyl Ester (1-14a)

To a solution of 1-13a (14.5 g, 31 mmol) in ethanol (150 mL) was added NaOH (31 mL of 1N solution in water, 31 mmol). After stirring at 0° C. for 30 minutes, the mixture was treated with HCl (31 mL of 1N solution in water, 1.56 mmol) and concentrated to give crude 1-14a.

### 3(S or R)-(2-Methoxy-pyrimidin-5-yl)-5-oxo-9-(6,7,8,9-tetrahydro-5H-pyrido[2,3-b]azepin-2-yl)-nonanoic Acid Ethyl Ester (1-15a)

The above crude residue was suspended in toluene (250 mL) and heated at reflux. After 1 h, HPLC showed complete conversion of the starting material to product 1-15a. Evaporation of the solvents gave crude 1-15a as a yellow oil.

### 3(S or R)-(2-Methoxy-pyrimidin-5-yl)-5-oxo-9-(6,7,8,9-tetrahydro-5H-pyrido[2,3-b]azepin-2-yl)-nonanoic Acid Ethyl Ester (1-16a)

To the above crude 1-15a in ethanol (100 mL) was added NaOH (32 mL of 1N solution in water, 32 mmol). After 1 hr, the mixture was concentrated, and the residue chromatographed on silica gel (25:10:1:1 to 15:10:1:1 ethyl acetate/ethanol/NH₄OH/water) to give 1-16a as a yellow solid (10.0 g, 76% from 1-13a).

R_{f}=0.21 (silica, 10:10:1:1 ethyl acetate/ethanol/NH₄OH/water).

¹H NMR (400 MHz, CDCl₃) δ 8.48 (s, 2H), 7.42 (d, 1H, J=7 Hz), 6.56 (d, 1H, J=7 Hz), 3.94 (s, 3H), 3.62 (m, 1H), 3.29 (m, 2H), 2.98 (m, 1H), 2.85 (m, 1H), 2.79 (m, 2H), 2.58 (m, 2H), 1.84 (m, 4H), 1.57 (m, 4H).

### 3(S or R)-(2-Methoxy-pyrimidin-5-yl)-5-oxo-9-(6,7,8,9-tetrahydro-5H-pyrido[2,3-b]azepin-2-yl)-nonanoic Acid Phosphoric Acid Salt (1-17a)

To a solution of 1-16a (0.169 g, 0.40 mmol) in absolute ethanol (3.0 mL) was added a solution of H₃PO₄ (50.3 mg of 85 wt. % solution in water, 0.44 mmol) in absolute ethanol (0.8 mL). The resulting cloudy solution was warmed to 60° C., causing complete dissolution, then allowed to cool to ambient temperature and stand for 15 hours. The solid was collected by filtration, washed with absolute ethanol (2 mL), and dried under a stream of nitrogen to give 1-17a as a light yellow solid (0.151 g, 72%); m.p. 127° C. Thermogravimetric analysis showed 1.2% weight loss of volatiles upon heating from 20 to 120° C.

¹H NMR (400 MHz, DMSO-d₆) δ 8.49 (s, 2H), 7.24 (d, 1H), 6.44 (d, 1H), 5.93 (br s, 1H), 3.85 (s, 3H), 3.43 (m, 1H), 3.02 (m, 2H), 2.83 (m, 2H), 2.64 (m, 2H), 2.55 (m, 2H), 2.42 (m, 2H), 2.38 (m, 2H), 2.64 (m, 4H), 1.43 (m, 4H). Analysis for C₂₃H₃₀N₄O₄.1.1H₃PO₄ or C₂₃H₃₀N₄O₄.1.0H₃PO₄.0.2H₂O: C=51.51%, H=5.89%, N=10.28%.

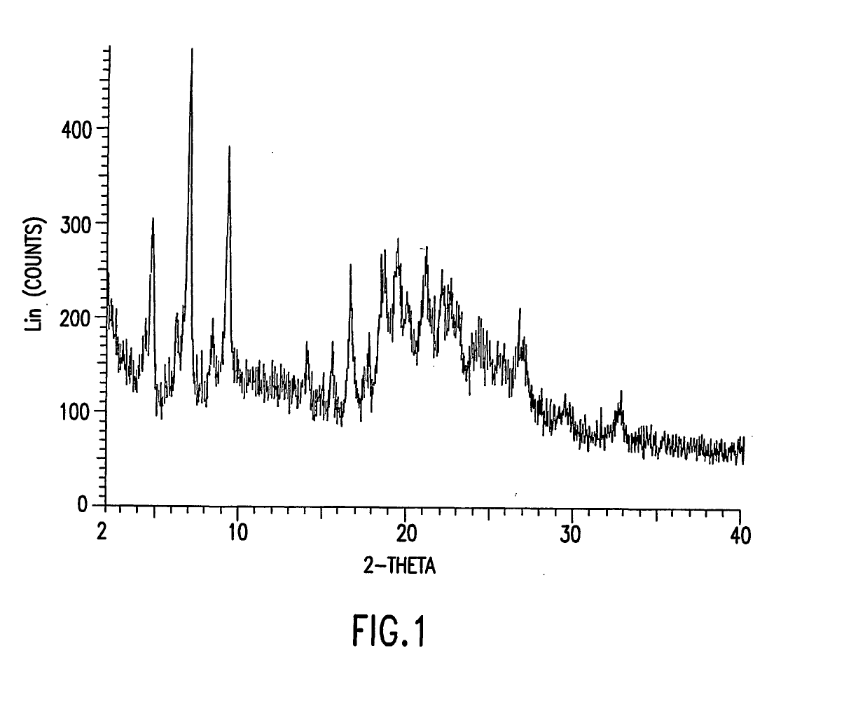

The X-ray powder diffraction pattern of the crystalline phosphoric salt is illustrated in FIG. 1. It has characteristic peaks at the following two theta values: 4.3°, 4.7°, 6.2°, 6.9°, 8.3°, 9.2°, 13.9°, 15.4°, 16.5°, 17.6°, 18.3°, 18.5°, 19.9°, 21.0°, 22.0°, 23.0°, and 32.6°. The X-ray pattern was obtained on a Siemens D5000 X-ray diffractometer, using Cu Kα radiation.

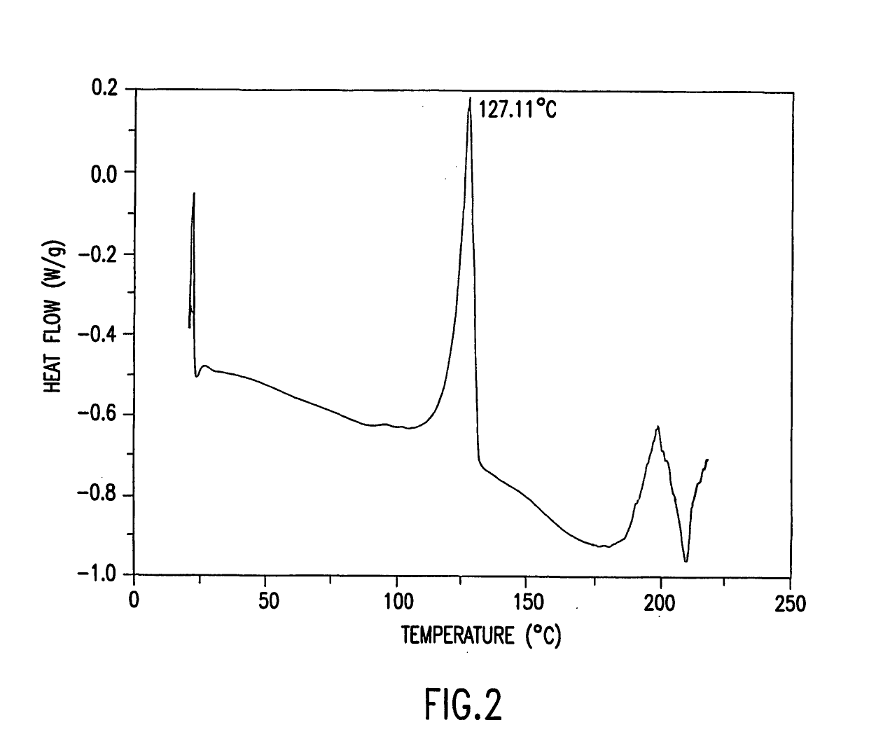

The differential scanning calorimeter (DSC) curve is illustrated in FIG. 2 and was taken on a TA 2920 Differential Scanning Calorimeter with a heating rate of 5° C./1 minute under nitrogen. The DSC curve exhibits a melting/decomposition endotherm with a peak temperature of about 127° C. (extrapolated onset temperature of about 120° C.).

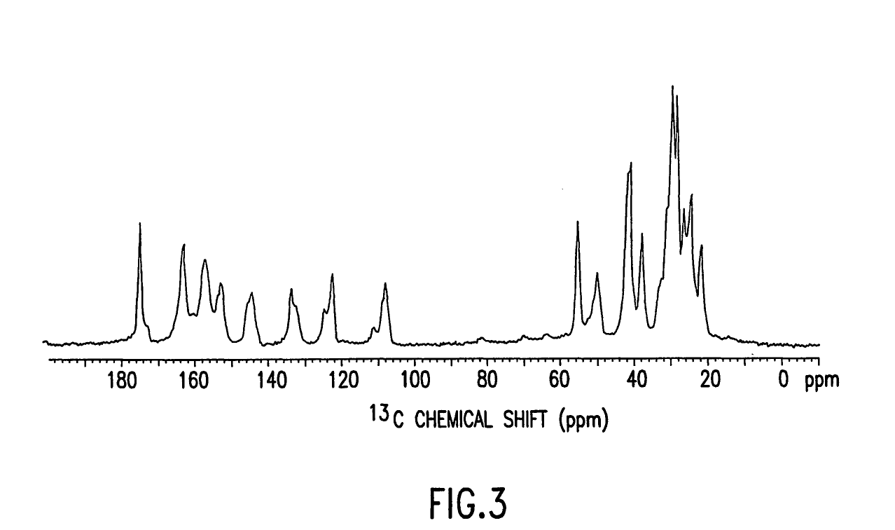

The crystalline phosphate salt was also characterized by solid-state NMR spectroscopy using a 200 MHz Varian Inova solid-state NMR spectrometer. FIG. 3 illustrates the carbon-13 CPMAS spectrum of the crystalline salt collected with a contact time of 1.5 seconds and a pulse delay of 5 seconds. The sample was spun at 4.8 kHz during the experiment. The spectrum exhibits signals with chemical shift values at 212.0, 174.5, 162.6, 156.6, 152.1, 143.9, 133.2, 124.0, 122.0, 110.9, 107.4, 55.4, 50.0, 41.1, 37.7, 29.7, 28.4, 26.6, 24.7, and 22.0 ppm.

### EXAMPLE 2

### 3(R or S)-(2-Methoxy-pyrimidin-5-yl)-5-oxo-9-(6,7,8,9-tetrahydro-5H-pyrido[2,3-b]azepin-2-yl)-nonanoic Acid Phosphoric Acid Salt (1-17b)

This phosphoric acid salt was prepared from 1-13b as described for 1-17a.

### EXAMPLES OF PHARMACEUTICAL FORMULATIONS

The phosphoric acid salt of formula I is formulated into a tablet by a direct compression process. A 100 mg potency tablet is composed of 123 mg of the active ingredient, 253 mg microcrystalline cellulose, 20 mg of croscarmellose sodium, and 4 mg of magnesium stearate. The active ingredient, microcrystalline cellulose, and croscarmellose are first blended, and the mixture is then lubricated with magnesium stearate and pressed into tablets.

An intravenous (i.v.) aqueous formulation is prepared by dissolving the phosphoric acid salt of formula I in normal saline. For a formulation with a concentration of 10 mg/mL, 1.23 mg of the phosphoric acid salt and 9 mg of sodium chloride are dissolved in one mL solution.

1. A salt of 3-(2-methoxy-pyrimidin-5-yl)-5-oxo-9-(6,7,8,9-tetrahydro-5H-pyrido[2,3-b]azepin-2-yl)-nonanoic acid of structural formula I: 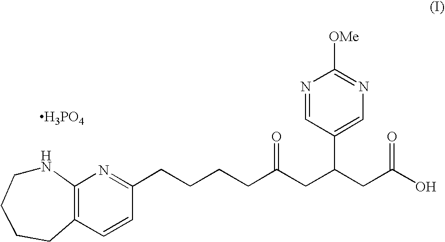
or a pharmaceutically acceptable solvate thereof.

2. The salt of claim 1 of structural formula II having the (S)-configuration at the chiral center marked with an *

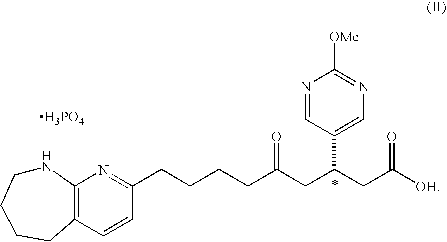

3. The salt of claim 1 of structural formula III having the (R) configuration at the chiral center marked with an *

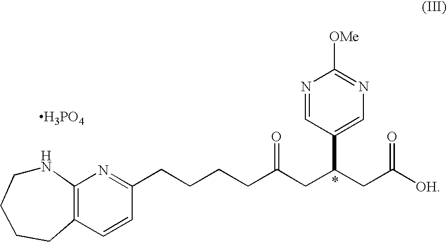

4. The crystalline salt of claim 1 characterized by the X-ray powder diffraction pattern of FIG. 1.

5. The crystalline salt of claim 1 characterized by the differential scanning calorimetric curve of FIG. 2.

6. The crystalline salt of claim 1 characterized by the solid-state carbon-13 nuclear magnetic resonance spectrum of FIG. 3.

7. A salt comprising the ions of monoprotonated 3-(2-methoxy-pyrimidin-5-yl)-5-oxo-9-(6,7,8,9-tetrahydro-5H-pyrido[2,3-b]azepin-2-yl)-nonanoic acid cation and dihydrogenphosphate anion.

8. A pharmaceutical composition comprising a prophylactically or therapeutically effective amount of the salt according to claim 1 or a pharmaceutically acceptable solvate thereof in association with one or more pharmaceutically acceptable carriers.

9. A method for the prevention and/or treatment of osteoporosis comprising administering to a patient in need of such prevention or treatment a prophylactically or therapeutically effective amount of the salt according to claim 1, or a pharmaceutically acceptable solvate thereof.

10. A method for the treatment of a disease or condition characterized by excessive angiogenesis comprising administering to a patient in need of such treatment a therapeutically effective amount of the salt according to claim 1, or a pharmaceutically acceptable solvate thereof.

11. The method of claim 8 wherein said disease or condition is selected from the group consisting of macular degeneration, vascular restenosis, diabetic retinopathy, atherosclerosis, inflammatory arthritis, cancer, and metastatic tumor growth.

12. A process for preparing the salt of claim 1 comprising the step of contacting one molar equivalent of 3-(2-methoxy-pyrimidin-5-yl)-5-oxo-9-(6,7,8,9-tetrahydro-5H-pyrido[2,3-b]azepin-2-yl)-nonanoic acid in an organic solvent with about a one molar equivalent of phosphoric acid at a temperature in the range of about 25-100° C.

13. The process of claim 12 wherein said organic solvent is a C₁-C₄ linear or branched alkanol.

14. Use of the salt of claim 1 as active ingredient in the manufacture of a medicament for use in the treatment and/or prevention of osteoporosis and for the treatment of vascular restenosis, macular degeneration, diabetic retinopathy, atherosclerosis, inflammatory arthritis, cancer, and metastatic tumor growth.

15. The pharmaceutical composition of claim 8 adapted for i.v. administration.

16. The salt of 3-(2-methoxy-pyrimidin-5-yl)-5-oxo-9-(6,7,8,9-tetrahydro-5H-pyrido[2,3-b]azepin-2-yl)-nonanoic acid prepared according to the process of claim 12.
